(19) United States
(12) Patent Application Publication
(10) Pub. No.: US 2026/0364884 A1
(43) Pub. Date: Nov. 19, 2026

(54) INSPECTION METHOD FOR UN-INSPECTABLE FEATURE OF ADDITIVE MANUFACTURED COMPONENT

(71) Applicant: GE Vernova Infrastructure Technology LLC, Greenville, SC (US)

(72) Inventors: John Bryan Pourcho, Simpsonville, SC (US); Clay Thomas Griffis, Mauldin, SC (US); Evan John Dozier, Greenville, SC (US); James Joseph Murray, Mauldin, SC (US); Maxwell Evan Miller, Simpsonville, SC (US)

(21) Appl. No.: 19/211,763

(22) Filed: May 19, 2025

(51) Int. Cl. B29C 64/386; B33Y 10/00; B33Y 50/00

(52) U.S. Cl. CPC B29C 64/386; B33Y 50/00; B33Y 10/00

(57) ABSTRACT

The methods may include additively manufacturing a feature test coupon including a test feature, where the test feature is a replica of a feature of the AM component that is un-inspectable, e.g., from an exterior. The feature test coupon is mounted on a fixture for holding the feature test coupon relative to an inspection system, and inspecting is performed on the test feature on the feature test coupon using the inspection system. Where a defect is detected in the test feature on the feature test coupon, a parameter of the additive manufacturing system and/or the feature is adjusted to remove the defect. The methods may also include configuring the feature test coupon and a fixture for holding the feature test coupon to maintain the feature on the feature test coupon within a focal field of the inspection system as the fixture is moved through a predefined inspection path.

### TECHNICAL FIELD

The disclosure relates generally to component inspection. More specifically, the disclosure relates to an inspection method for a feature of an additive manufactured component that is un-inspectable.

### BACKGROUND

Additive manufacturing (AM) includes a wide variety of processes of producing a component through the successive layering of material rather than the removal of material. As such, additive manufacturing can create complex geometries without the use of any sort of tools, molds or fixtures, and with little or no waste material. Instead of machining components from solid billets of material, much of which is cut away and discarded, the only material used in additive manufacturing is what is required to shape the component. Additive manufacturing techniques typically include taking a three-dimensional computer aided design (CAD) file of the component to be formed, electronically slicing the component into layers, e.g., 18-102 micrometers thick, and creating a file with a two-dimensional image of each layer, including vectors, images or coordinates. The file may then be loaded into a preparation software system that interprets the file such that the component can be built by different types of additive manufacturing systems.

Current inspection approaches build the AM component and then mount it in a unique and complex fixture for inspection by, for example, a multi-scan inspection system. The multi-scan inspection system may use, for example, visual, color vision (e.g., blue-light, laser, infrared, etc.), shape analysis and/or X-ray inspection methods. The multi-scan inspection system is programmed to move the AM component and inspect external features thereof. When a flaw is identified in an (inspectable) external feature, operating parameter(s) of the additive manufacturing system are adjusted and the AM component is re-made. The process may then be repeated to ensure the feature is properly made.

One challenge with evaluating an AM component is some features thereof are very small or fine and difficult to inspect on a much larger AM component. In one non-limiting example, a very small or fine feature may be in the range of 0.600 millimeters (mm) (~0.024 inches(in.)) +/−0.0762 mm (0.003 in.). One example includes a small fuel nozzle for a combustor component for a turbomachine. For context, an inner diameter of the opening in the fuel nozzle may be in the range of 0.5-0.7 millimeters or smaller. The additively manufactured micromixer in which it is located may be 50 centimeters long and 20 centimeters deep and weigh 2.2 kilograms. Current approaches mount the large AM component in a custom-built and expensive fixture for an inspection system. This approach may requires more than one fixture to inspect all of the small features of the entire AM component, adjustment of the multi-scan inspection system software, and/or adjustment of the overall automated inspection process. This approach is expensive and time-consuming and oftentimes does not result in high quality data regarding the small or fine features. Smaller features of an AM component may also be incapable of certain types of inspection, such as a computed tomography (CT) scan.

Another challenge with evaluating an AM component is some features thereof are not inspectable from an exterior of the AM component. A feature may be un-inspectable because there is some limitation on the line-of-sight to the feature, e.g., the feature is hidden on or within the AM component. Addressing the line-of-sight issues may require (possibly repeated) additive manufacture and destruction of the AM component to better expose the feature, use of more than one fixture to hold the AM component, adjustment of the multi-scan inspection system software, and/or adjustment of the overall automated inspection process. All of these approaches add expense and time to the inspection process.

### BRIEF DESCRIPTION

All aspects, examples and features mentioned below can be combined in any technically possible way.

An aspect of the disclosure provides a method, comprising: first additively manufacturing a feature test coupon including a test feature, the test feature being a replica of a feature of an additively manufactured (AM) component formed by an additive manufacturing system that is un-inspectable; mounting the feature test coupon on a fixture for holding the feature test coupon relative to an inspection system; inspecting the test feature on the feature test coupon using the inspection system; and in response to a defect being detected in the test feature on the feature test coupon, adjusting a parameter of at least one of the additive manufacturing system or the feature to remove the defect.

Another aspect of the disclosure includes any of the preceding aspects and the feature is un-inspectable from an exterior of an additively manufactured (AM) component formed by the additive manufacturing system.

Another aspect of the disclosure includes any of the preceding aspects, and further comprising: identifying the feature of the AM component that is within the AM component to be inspected; and configuring the feature test coupon including the test feature and the fixture for holding the feature test coupon relative to the inspection system to maintain the test feature on the feature test coupon within a focal field of the inspection system as the fixture is moved through a predefined inspection path.

Another aspect of the disclosure includes any of the preceding aspects, and the focal field includes a three-dimensional window in which focusing of any inspection element of the inspection system is not required.

Another aspect of the disclosure includes any of the preceding aspects, and the feature test coupon is configured to orient the test feature for inspection of a set of predefined critical-to-quality (CTQ) parameters.

Another aspect of the disclosure includes any of the preceding aspects, and the feature test coupon includes a first mating element configured to removably mate with a second mating element of the fixture in a manner to present a first portion of the test feature for the inspecting.

Another aspect of the disclosure includes any of the preceding aspects, and the feature test coupon includes a third mating element configured to removably mate with the second mating element of the fixture in a manner to present a second, different portion of the test feature for the inspecting.

Another aspect of the disclosure includes any of the preceding aspects, and the predefined inspection path includes at least one linear movement in at least one of an X, Y and Z direction, tilt around at least one of an X and Y axis, and rotation around a Z axis.

Another aspect of the disclosure includes any of the preceding aspects, and the first additively manufacturing the feature test coupon includes additively manufacturing a plurality of the feature test coupons, wherein the test feature on each of the plurality of feature test coupons has a different characteristic than the test feature on each other of the plurality of feature test coupons, and wherein the inspecting includes inspecting each test feature on the plurality of feature test coupons using the inspection system, and wherein the adjusting includes, in response to the defect being detected in the test feature on at least one of the plurality of feature test coupons, adjusting the parameter to remove the defect.

Another aspect of the disclosure includes any of the preceding aspects, and further comprising a second additively manufacturing step performed with the parameter of the least one of additive manufacturing system or the feature being different from the first additively manufacturing step, the second additively manufacturing step manufacturing the feature test coupon including an adjusted test feature.

Another aspect of the disclosure includes any of the preceding aspects, and further comprising repeating the additive manufacturing on the additive manufacturing system, mounting and inspecting steps using an adjusted test feature.

Another aspect of the disclosure includes any of the preceding aspects, and the test feature that is un-inspectable from the exterior of the AM component is located within the AM component.

Another aspect of the disclosure includes a method, comprising: identifying a feature of an additively manufactured (AM) component formed by an additive manufacturing system that is un-inspectable; configuring a feature test coupon including a test feature that is substantially a replica of the feature of the AM component and a fixture for holding the feature test coupon relative to an inspection system to maintain the test feature on the feature test coupon within a focal field of the inspection system as the fixture is moved through a predefined inspection path; first additively manufacturing the feature test coupon on the additive manufacturing system; mounting the fixture relative to the inspection system and the feature test coupon on the fixture; inspecting the test feature on the feature test coupon using the inspection system and moving the fixture along the predefined inspection path to identify a defect in the test feature on the feature test coupon, wherein the test feature on the feature test coupon remains within the focal field of the inspection system as the fixture is moved through the predefined inspection path; and in response to a defect being detected in the test feature on the feature test coupon, adjusting a parameter of at least one of the additive manufacturing system or the feature to remove the defect.

Another aspect of the disclosure includes any of the preceding aspects, and the feature is located within the AM component.

Another aspect of the disclosure includes any of the preceding aspects, and the focal field includes a three-dimensional window in which focusing of any inspection element of the inspection system is not required.

Another aspect of the disclosure includes any of the preceding aspects, and the additive manufacturing of the feature test coupon is configured to orient the test feature for inspection of a set of predefined critical-to-quality (CTQ) parameters.

Another aspect of the disclosure includes any of the preceding aspects, and the feature test coupon includes a first mating element configured to removably mate with a second mating element of the fixture in a manner to present a first portion of the test feature for the inspecting.

Another aspect of the disclosure includes any of the preceding aspects, and the feature test coupon includes a third mating element configured to removably mate with the second mating element of the fixture in a manner to present a second, different portion of the test feature for the inspecting.

Another aspect of the disclosure includes any of the preceding aspects, and the predefined inspection path includes at least one linear movement in at least one of an X, Y and Z direction, tilt around at least one of an X and Y axis, and rotation around a Z axis.

Another aspect of the disclosure includes any of the preceding aspects, and further comprising a second additively manufacturing step performed with the parameter of the at least one of the additive manufacturing system or the feature being different from the first additively manufacturing step, the second additively manufacturing step manufacturing the feature test coupon including an adjusted test feature.

Another aspect of the disclosure includes any of the preceding aspects, and further comprising repeating the first additively manufacturing step, mounting and inspecting using an adjusted test feature.

Two or more aspects described in this disclosure, including those described in this summary section, may be combined to form implementations not specifically described herein. That is, all embodiments described herein can be combined with each other.

The details of one or more implementations are set forth in the accompanying drawings and the description below. Other features, objects and advantages will be apparent from the description and drawings, and from the claims.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 shows a cross-sectional view of an illustrative additively manufactured component including a feature for inspection, according to embodiments of the disclosure;

FIG. 2 shows a perspective view of a feature test coupon including a replica of a feature to be inspected, according to embodiments of the disclosure;

FIG. 3 shows a perspective view of a feature test coupon in a fixture for an inspection system, according to embodiments of the disclosure;

FIG. 4 shows a flow diagram of a method of inspecting a feature of an additively manufactured component, according to embodiments of the disclosure;

FIG. 5 shows a schematic view of an illustrative additive manufacturing system, according to embodiments of the disclosure;

FIG. 6 shows a cross-sectional view of a plurality of parallel, sintered metal layers of a feature test coupon or an additively manufactured component, according to embodiments of the disclosure;

FIG. 7 shows a perspective view of an illustrative inspection system used in methods according to embodiments of the disclosure;

FIG. 8 shows a cross-sectional view of a feature test coupon in a fixture for an inspection system, according to embodiments of the disclosure;

FIG. 9 shows a perspective view of a feature test coupon in a fixture for an inspection system, according to embodiments of the disclosure;

FIG. 10 shows a perspective view of a feature test coupon in a fixture for an inspection system, according to other embodiments of the disclosure;

FIG. 11 shows a flow diagram of a method of inspecting a feature of an additively manufactured component, according to other embodiments of the disclosure;

FIG. 12 shows a side view of a fixture with a feature test coupon and an inspection head and a platen of an inspection system, according to embodiments of the disclosure;

FIG. 13 shows a side view of a fixture with a feature test coupon and an inspection head and a platen of an inspection system, according to embodiments of the disclosure;

FIG. 14 shows a side view of a fixture with a feature test coupon and an inspection head and a platen of an inspection system, according to embodiments of the disclosure;

FIG. 15 shows a perspective view of a feature test coupon including a replica of a feature to be inspected, according to other embodiments of the disclosure;

FIG. 16 shows a perspective view of the feature test coupon of FIG. 15 in a mount of a fixture for an inspection system, according to other embodiments of the disclosure;

FIG. 17 shows a perspective view of a feature test coupon including a replica of a feature to be inspected, according to additional embodiments of the disclosure;

FIG. 18 shows a perspective view of the feature test coupon of FIG. 17 in a mount of a fixture for an inspection system, according to additional embodiments of the disclosure; and

FIG. 19 shows a perspective view of a feature test coupon including a replica of a feature to be inspected, according to other embodiments of the disclosure.

It is noted that the drawings of the disclosure are not necessarily to scale. The drawings are intended to depict only typical aspects of the disclosure and therefore should not be considered as limiting the scope of the disclosure. In the drawings, like numbering represents like elements between the drawings.

### DETAILED DESCRIPTION

As an initial matter, in order to clearly describe the subject matter of the current technology, it will become necessary to select certain terminology when referring to and describing relevant machine components within the illustrative application of a turbine component. When doing this, if possible, common industry terminology will be used and employed in a manner consistent with its accepted meaning. Unless otherwise stated, such terminology should be given a broad interpretation consistent with the context of the present application and the scope of the appended claims. Those of ordinary skill in the art will appreciate that often a particular component may be referred to using several different or overlapping terms. What may be described herein as being a single part may include and be referenced in another context as consisting of multiple components. Alternatively, what may be described herein as including multiple components may be referred to elsewhere as a single part.

It is often required to describe parts that are at different radial positions with regard to a center axis. The term “axial” refers to movement or position parallel to an axis, e.g., an axis of a turbomachine. The term “radial” refers to movement or position perpendicular to an axis, e.g., an axis of a turbomachine. In cases such as this, if a first component resides closer to the axis than a second component, it will be stated herein that the first component is “radially inward” or “inboard” of the second component. If, on the other hand, the first component resides further from the axis than the second component, it may be stated herein that the first component is “radially outward” or “outboard” of the second component. Finally, the term “circumferential” refers to movement or position around an axis, e.g., a circumferential interior surface of a casing extending about an axis of a turbomachine. As indicated above, it will be appreciated that such terms may be applied in relation to the axis of the turbomachine.

In addition, several descriptive terms may be used regularly herein, as described below. The terms “first,” “second,” and “third,” may be used interchangeably to distinguish one component from another and are not intended to signify location or importance of the individual components.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event may or may not occur or that the subsequently described feature may or may not be present and that the description includes instances where the event occurs or the feature is present and instances where the event does not occur or the feature is not present.

Where an element or layer is referred to as being “on,” “engaged to,” “connected to,” “coupled to,” or “mounted to” another element or layer, it may be directly on, engaged, connected, coupled, or mounted to the other element or layer, or intervening elements or layers may be present. In contrast, when an element is referred to as being “directly on,” “directly engaged to,” “directly connected to,” or “directly coupled to” another element or layer, there are no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items. The verb forms of “couple” and “mount” may be used interchangeably herein.

Embodiments of the disclosure provide methods to address inspection of a feature that is un-inspectable from an exterior of an additively manufactured (AM) component. The methods may include additively manufacturing a feature test coupon including a test feature, where the test feature is a replica of a feature of the AM component formed by an additive manufacturing system that is un-inspectable, e.g., from an exterior of the AM component. The methods also include mounting the feature test coupon on a fixture for holding the feature test coupon relative to an inspection system and inspecting the test feature on the feature test coupon using the inspection system. Where a defect is detected in the test feature on the feature test coupon, the method includes adjusting a parameter of at least one of the additive manufacturing system or the feature to remove the defect. In certain embodiments, the method may additionally, or alternatively, include configuring the feature test coupon including the feature that is a replica of a feature the AM component that is un-inspectable and a fixture for holding the feature test coupon relative to the inspection system to maintain the feature on the feature test coupon within a focal field of the inspection system as the fixture is moved through a predefined inspection path.

The methods provide for inspecting features that are un-inspectable to be reliably, inexpensively, or efficiently inspected utilizing conventional methods. The methods are advantageous for features that are un-inspectable, for example, from an exterior of the AM component or are too small or fine features that are not easily inspectable on a large AM component. The feature test coupon may be standardized and fits into a specifically designed fixture that places the feature within the focal field of the inspection system. The inspection may include, for example, optical surface roughness and shape measuring, with an automated multi-scan inspection system that combines multiple fine surface roughness scans into a high resolution, single geometry output for development and ongoing quality checks. Isolating the small features that are not otherwise inspectable onto a universal feature test coupon also increases the abilities of the non-destructive inspection over similar blue-light technology. An automated multi-scan inspection system allows for consistent, reliable results at the resolution of a surface roughness measurement. The methods also allow for rapid feedback thus allowing for both operational parameter and geometric parameter improvements. The methods can be used regardless of component or feature geometry, materials, or AM system used. The efficient, automated and consistent inspections provide quantitative results for both the component (short-term) and the machine (long-term) in which the feature is used, e.g., a turbomachine.

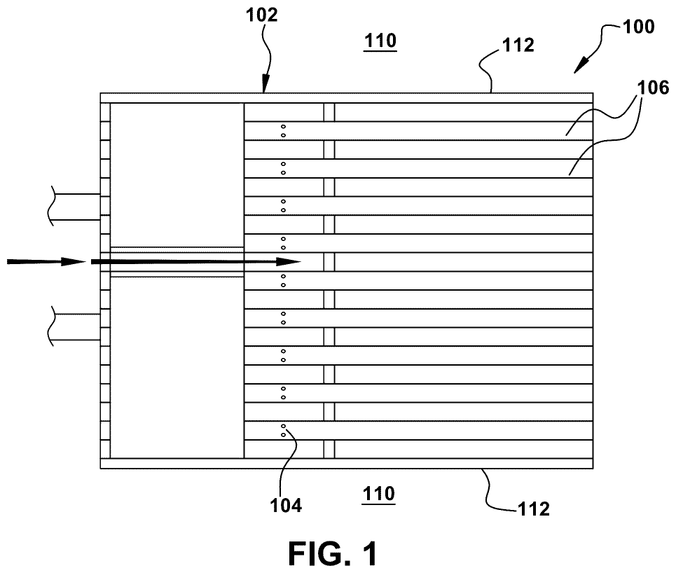

FIG. 1 shows a cross-sectional view of an illustrative additively manufactured (AM) component 100. AM component 100 is additively manufactured and may include any industrial component. In the example shown, AM component 100 includes a micromixer 102 for a combustor of a turbomachine. AM component 100 includes a feature 104 that is un-inspectable, e.g., from an exterior 110 of AM component 100. Feature 104 may be, for example, on exterior surface 112 of AM component 100 but hidden behind another part of AM component 100. Alternatively, as shown in the example in FIG. 1, feature 104 that is un-inspectable from exterior 110 of AM component 100 may be located within AM component 100, e.g., within exterior surface 112 of AM component 100. In other embodiments, feature 104 may also be any small or fine feature anywhere on AM component 100 that is difficult to inspect. For example, feature 104 may be very small, e.g., in the range of 0.600 millimeters (mm) (~0.024 inches(in.)) +/−0.0762 mm (0.003 in.). For purposes of description, feature 104 includes a small fuel opening in a mixing tube 106 of micromixer 102, e.g., a circular opening having a beveled inner and outer edge, that can be within micromixer 102. Feature 104 is significantly smaller than the overall AM component 100. For context, an inner diameter of the fuel opening may be in the range of 0.5-0.7 millimeters or smaller, and micromixer 102 may be 50 centimeters long, 20 centimeters deep and weigh 2.2 kilograms. Feature 104 can take the form of any portion of AM component 100 for which inspection is not possible from exterior 110 of AM component 100. A non-comprehensive list of possible features 104 of micromixer 102 to be inspected according to embodiments of the disclosure may include: fuel nozzle or hole; cooling or air hole or channel; heat transfer structures like bumps, bosses, ribs, etc.; cracks in critical internal features; support structures; stand-offs between internal walls; tear drop parameter development for holes; thin wall conditions; and/or small complex cooling shape. It is emphasized that other AM components 100 may have different features 104 to be inspected. Accordingly, it will be recognized that feature 104 can take a variety of alternative forms than that disclosed herein.

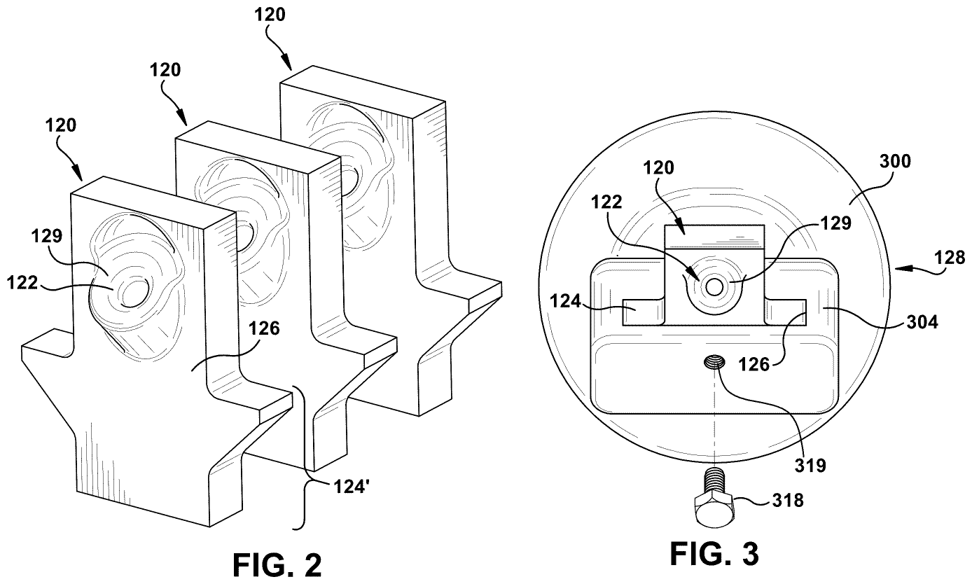

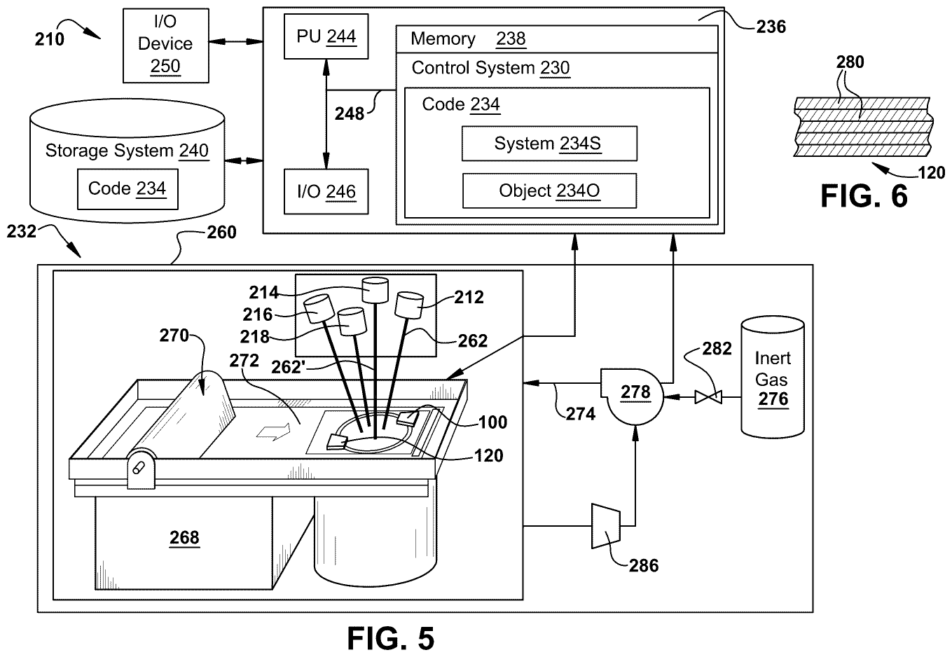

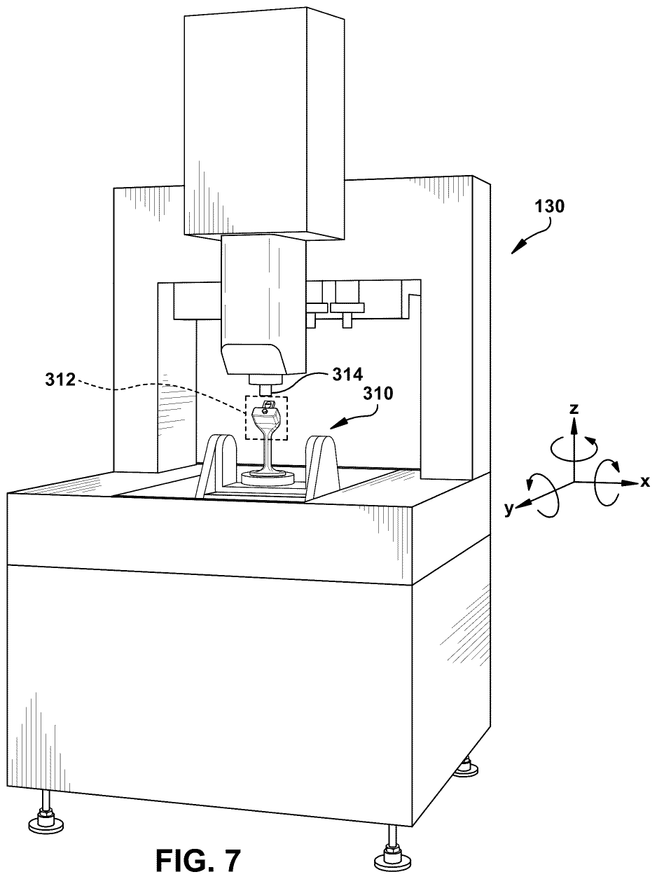

FIG. 2 shows an enlarged perspective view of a feature test coupon 120 including a test feature 122. Test feature 122 is a replica of a feature of an additive manufacturing (AM) system 210 (FIG. 5, described further herein) that is un-inspectable. That is, test feature 122 is an exact copy of a feature to be inspected, e.g., feature 104 (FIG. 1). The extent of feature 104 that forms test feature 122 can be user selected to ensure the desired data from the inspection can be obtained. In this manner, feature test coupon 120 can easily isolate any fine feature 104 and presents a flexible solution to fit any design criteria. In certain embodiments, the feature is un-inspectable from exterior 110 (FIG. 1) of AM component 100 (FIG. 1) formed by AM system 210 (FIG. 5), but this may not be the situation in all cases. In the example shown in FIGS. 1 and 2, feature 104 and test feature 122 includes a fuel opening in mixing tube 106. As used herein, feature 104 may be un-inspectable because there is some limitation on the line-of-sight to the feature, e.g., the feature is hidden on or within AM component 100. In addition, a feature 104 may be un-inspectable because it is too small or fine relative to AM component 100 to be inspected in a quality manner due to, for example, limitations on the line-of-sight to the feature or AM component 100 size is too large to allow the small features to be inspected. Limitations in the line-of-sight to the small feature can arise in a number of ways. For example, AM component 100 may be too large to fit within inspection system 130 (FIG. 7). In other cases, AM component 100 is too large to be moved relative to an inspection system 130 (FIG. 7) in a manner to inspect the small features thereon, or it may require complex, unique fixturing in order to be inspected. Alternatively, feature 104 may be behind or hidden behind another structure of AM component 100. In the example shown, feature 104 is located within an exterior surface 112 of AM component 100. In other cases, feature may be very small, e.g., in the range of 0.600 millimeters (mm) (~0.024 inches(in.)) +/−0.0762 mm (0.003 in.). Line-of-sight issues can also arise when rapid prototyping fine features for geometry, material analysis, additive laser property troubleshooting, and/or production quality checks.

Feature test coupon 120 also may include a variety of structures for mounting feature test coupon 120 to a fixture 128 for inspection system 130 (FIG. 7). In certain embodiments, feature test coupon 120 includes a first mating element 124 configured to removably mate with a second mating element 126 of (mount 304) of fixture 128 in a manner to present a first portion 129 of test feature 122 for inspecting. Further options for feature test coupon 120 will be described herein. As will also be described further herein, feature test coupon 120 may be configured to orient test feature 122 in a variety of ways to allow inspection of desired portions and/or characteristics of test feature 122. For example, feature test coupon 120 may be configured to orient test feature 122 for inspection of a set of predefined critical-to-quality (CTQ) parameters. The CTQ parameters may be different depending on the form of test feature 122, the portion thereof to be inspected, and/or the desired data to be collected. A non-comprehensive list of CTQ parameters includes surface roughness; shape; dimension(s) and/or tolerances; location; material properties; cracking; yield; and/or process consistency.

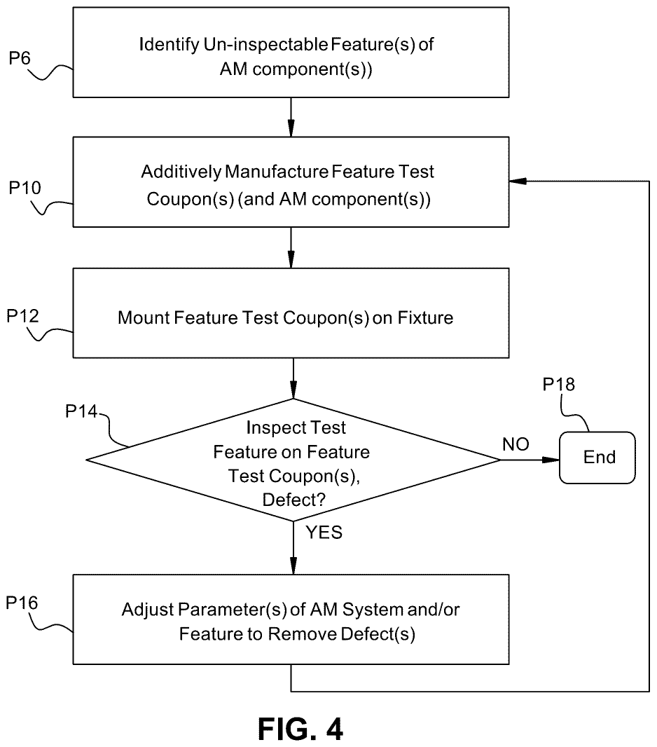

FIG. 4 shows a flow diagram of methods according to embodiments of the disclosure. With reference to FIGS. 1, 3 and 7-10, in optional process P6, feature 104 of AM component 100 formed by AM system 210 (FIG. 5) that is un-inspectable is identified. In certain embodiments, features 104 may be located within AM component 100, i.e., within exterior surface 112 of AM component 100. However, feature 104 can take any un-inspectable form described herein. Feature 104 so identified may include any feature described herein benefiting from the methods described herein. Feature 104 may be identified using any now known or later developed solution, e.g., user selection, modeling, empirical data, etc. The extent of feature 104 that forms test feature 122 can be user selected to ensure the desired data from the inspection can be obtained.

Process P10 includes (first) additively manufacturing feature test coupon 120 including test feature 122. As noted, test feature 122 is a replica of feature 104 (FIG. 1) of AM component 100 (FIG. 1) formed by AM system 210 (FIG. 5) that is un-inspectable, e.g., from exterior 110 (FIG. 1) of AM component 100 (FIG. 1).

FIG. 5 shows a schematic/block diagram of an illustrative computerized metal powder additive manufacturing system 210 (hereinafter ‘AM system 210’) for generating feature test coupon 120 (and separately perhaps AM component 100), of which only a single layer is shown. While feature test coupon 120 and AM component 100 are shown being printed together, they may be printed separately. The teachings of the disclosures will be described relative to building feature test coupon 120 (and AM component 100) using multiple melting beam sources 212, 214, 216, 218, but it is emphasized and will be readily recognized that the teachings of the disclosure are equally applicable to build feature test coupon 120 using any number of melting beam sources. In this example, AM system 210 is arranged for direct metal laser melting (DMLM). It is understood that the general teachings of the disclosure are equally applicable to other forms of metal powder additive manufacturing such as but not limited to selective laser melting (SLM), and perhaps other forms of additive manufacturing (i.e., other than metal powder applications). In addition, it is understood that the general teachings of the disclosure are equally applicable to other forms of additive manufacturing. The layer of feature test coupon 120 and AM component 100 in build platform 220 are illustrated as a circular or square element, respectively, in FIG. 5; however, it is understood that the additive manufacturing process can be readily adapted to manufacture any shape on build platform 220, e.g., that of feature test coupon 102 and/or AM component 100.

AM system 210 generally includes an additive manufacturing control system 230 (“control system”) and an AM printer 232. As will be described, control system 230 executes a set of computer-executable instructions or code 234 to generate feature test coupon 120 or AM component 100 using multiple melting beam sources 212, 214, 216, 218. In the example shown, four melting beam sources may include four lasers. However, the teachings of the disclosures are applicable to any melting beam source, e.g., an electron beam, laser, etc. Control system 230 is shown implemented on computer 236 as computer program code. To this extent, computer 236 is shown including a memory 238 and/or storage system 240, a processor unit (PU) 244, an input/output (I/O) interface 246, and a bus 248. Further, computer 236 is shown in communication with an external I/O device/resource 250. In general, processor unit (PU) 244 executes computer program code 234 that is stored in memory 238 and/or storage system 240. While executing computer program code 234, processor unit (PU) 244 can read and/or write data to/from memory 238, storage system 240, I/O device 250 and/or AM printer 232. Bus 248 provides a communication link between each of the components in computer 236, and I/O device 250 can comprise any device that enables a user to interact with computer 236 (e.g., keyboard, pointing device, display, etc.).

Computer 236 is only representative of various possible combinations of hardware and software. For example, processor unit (PU) 244 may comprise a single processing unit or be distributed across one or more processing units in one or more locations, e.g., on a client and server. Similarly, memory 238 and/or storage system 240 may reside at one or more physical locations. Memory 238 and/or storage system 240 can comprise any combination of various types of non-transitory computer readable storage medium including magnetic media, optical media, random access memory (RAM), read only memory (ROM), etc. Computer 236 can comprise any type of computing device such as an industrial controller, a network server, a desktop computer, a laptop, a handheld device, etc.

As noted, AM system 210 and, in particular control system 230, executes code 234 to generate the desired object. Code 234 can include, among other things, a set of computer-executable instructions 234S (herein also referred to as ‘code 234S’) for operating a system (i.e., AM printer 232) and a set of computer-executable instructions 234O (herein also referred to as ‘code 234O’) for defining an object (e.g., feature test coupon 120 and/or AM component 100) to be physically generated by AM printer 232. As described herein, additive manufacturing processes begin with a non-transitory computer readable storage medium (e.g., memory 238, storage system 240, etc.) storing code 234. Set of computer-executable system instructions 234S for operating AM printer 232 may include any now-known or later developed software code capable of operating AM printer 232.

The set of computer-executable instructions 234O defining feature test coupon 120 (and perhaps separately AM component 100) may include a precisely defined 3D model of feature test coupon 120 (and/or AM component 100) and can be generated from any of a large variety of well-known computer aided design (CAD) software systems such as AutoCAD®, TurboCAD®, DesignCAD 3D Max, etc. In this regard, code 234O can include any now known or later developed file format. Furthermore, code 234O representative of the part to be built, e.g., feature test coupon 120 and/or AM component 100, may be translated between different formats. For example, code 234O may include Standard Tessellation Language (STL) files which was created for stereolithography CAD programs of 3D Systems, or an additive manufacturing file (AMF), which is an American Society of Mechanical Engineers (ASME) standard that is an extensible markup-language (XML) based format designed to allow any CAD software to describe the shape and composition of any three-dimensional object to be fabricated on any AM printer. Code 234O representative of feature test coupon 120 (and/or AM component 100) may also be converted into a set of data signals and transmitted, received as a set of data signals and converted to code, stored, etc., as necessary. Code 234O may be configured according to embodiments of the disclosure to allow for formation of border and internal sections in overlapping field regions, as will be described. In any event, code 234O may be an input to AM system 210 and may come from a part designer, an intellectual property (IP) provider, a design company, the operator or owner of AM system 210, or from other sources. In any event, control system 230 executes code 234S and 234O, dividing feature test coupon 120 (and separately perhaps AM component 100) into a series of thin slices that assembles using AM printer 232 in successive layers of material.

AM printer 232 may include a processing chamber 260 that is sealed to provide a controlled atmosphere for object printing. A build platform 220, upon which feature test coupon 120 is/are built, is positioned within processing chamber 260. A number of melting beam sources 212, 214, 216, 218 are configured to melt layers of metal powder on build platform 220 to generate feature test coupon 120 (and separately perhaps AM component 100). While four melting beam sources 212, 214, 216, 218 are illustrated, it is emphasized that the teachings of the disclosure are applicable to a system employing any number of sources, e.g., 1, 2, 3, or 5 or more. As understood in the field, each melting beam source 212, 214, 216, 218 may have a field including a non-overlapping field region, respectively, in which it can exclusively melt metal powder, and may include at least one overlapping field region in which two or more sources can melt metal powder. In this regard, each melting beam source 212, 214, 216, 218 may generate a melting beam, respectively, that fuses particles for each slice, as defined by code 234O. For example, in FIG. 5, melting beam source 212 is shown creating a layer of feature test coupon 120 (and AM component 100) using melting beam 262 in one region, while melting beam source 214 is shown creating a layer of feature test coupon 120 using melting beam 262′ in another region. Each melting beam source 212, 214, 216, 218 is calibrated in any now known or later developed manner. That is, each melting beam source 212, 214, 216, 218 has had its laser or electron beam's anticipated position relative to build platform 220 correlated with its actual position in order to provide an individual position correction (not shown) to ensure its individual accuracy. In one embodiment, each of plurality melting beam sources 212, 214, 216, 218 may create melting beams, e.g., 262, 262′, having the same cross-sectional dimensions (e.g., shape and size in operation), power and scan speed.

Continuing with FIG. 5, an applicator (or re-coater blade) 270 may create a thin layer of raw material 272 spread out as the blank canvas from which each successive slice of the final feature test coupon 120 (and/or AM component 100) will be created. Various parts of AM printer 232 may move to accommodate the addition of each new layer, e.g., a build platform 220 may lower and/or chamber 260 and/or applicator 270 may rise after each layer. The process may use different raw materials in the form of fine-grain metal powder, a stock of which may be held in a chamber 268 accessible by applicator 270.

Processing chamber 260 is filled with an inert gas such as argon or nitrogen and controlled to reduce or eliminate oxygen. Control system 230 is configured to control a flow of a gas mixture 274 within processing chamber 260 from a source of inert gas 276. In this case, control system 230 may control a pump 280, and/or a flow valve system 282 for inert gas to control the content of gas mixture 274. Flow valve system 282 may include one or more computer controllable valves, flow sensors, temperature sensors, pressure sensors, etc., capable of precisely controlling flow of the particular gas. Pump 280 may be provided with or without valve system 282. Where pump 280 is omitted, inert gas may simply enter a conduit or manifold prior to introduction to processing chamber 260. Source of inert gas 276 may take the form of any conventional source for the material contained therein, e.g., a tank, reservoir or other source. Any sensors (not shown) required to measure gas mixture 274 may be provided. Gas mixture 274 may be filtered using a filter 286 in a conventional manner.

In operation, build platform 220 with metal powder thereon is provided within processing chamber 260, and control system 230 controls flow of gas mixture 274 within processing chamber 260 from source of inert gas 276. Control system 230 also controls AM printer 232, and in particular, applicator 270 and melting beam sources 212, 214, 216, 218 to sequentially melt layers of metal powder on build platform 220 to generate the desired part according to embodiments of the disclosure. While a particular AM system 210 has been described herein, it is emphasized that the teachings of the disclosure are not limited to any particular additive manufacturing system or additive manufacturing method.

As shown in FIG. 6, due to the additive manufacture, feature test coupon 120 (and AM component 100) includes a plurality of parallel, sintered metal layers 280.

Returning to FIG. 4, and with reference to FIG. 3, process P12 includes mounting feature test coupon 120 on fixture 128 for holding feature test coupon 120 relative to an inspection system 130 (FIG. 7).

FIG. 7 shows a perspective view of an illustrative inspection system 130. Inspection system 130 may include any now known or later developed system for inspecting an industrial component. For example, inspection system 130 may include a multi-scan inspection system capable of visual, color vision (e.g., blue-light, laser, infrared, etc.), shape analysis and/or X-ray inspection methods. In one non-limiting example, inspection system 130 includes any appropriate model of optical 3D metrology and surface roughness measurement inspection systems available from Bruker Alicona®. Rather than a computer aided-design (CAD) model of AM component 100 being input to the CAD-based automatic multi-scan software of inspection system 130, a CAD model of test feature 122 is input, which narrows the focus of the algorithm to test feature 122 and results in higher quality data regarding the feature. Inspection system 130 performs a comparison of the CAD model of test feature 122 to that printed by AM system 210 (FIG. 5). As understood in the art, inspection system 130 is programmed to move feature test coupon 120 through any desired pre-programmed inspection tests and/or path to inspect test feature 122 thereof. The inspection may include any now known or later developed inspections(s). In one non-limiting example, the inspection may include optical surface roughness and shape measuring, that combines multiple fine surface roughness scans into a high resolution, single geometry output for development and ongoing quality checks. Inspection system 130 may include any number of inspection heads 314 to carry out different inspection types that are movable into or out of position to inspect feature test coupon 120 in focal field 312. The inspecting process does not require a specific inspection program and can be accomplished manually using single scans as long as the surface roughness output for each scan is geometric data and can be stitched together by an inspection software in analysis. In certain embodiments, inspection system 130 is capable of microscopic evaluation of feature test coupon 120 and test feature 122 thereof; however, this dimensional focus level may not be necessary in all cases. Inspection system 130 also includes 3D motion control system 310 capable of moving feature test coupon 120 through a predefined inspection path. As shown by the legend in FIG. 7, the predefined inspection path may include at least one linear movement in at least one of an X, Y and Z direction, tilt around at least one of an X and Y axis, and rotation around a Z axis. As the operation of inspection system 130 is well known in the art no further details of operation thereof is warranted.

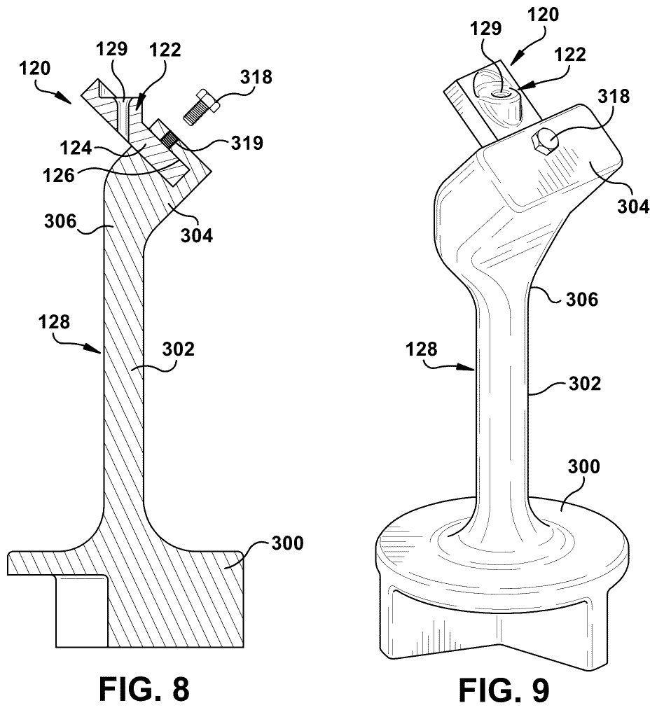

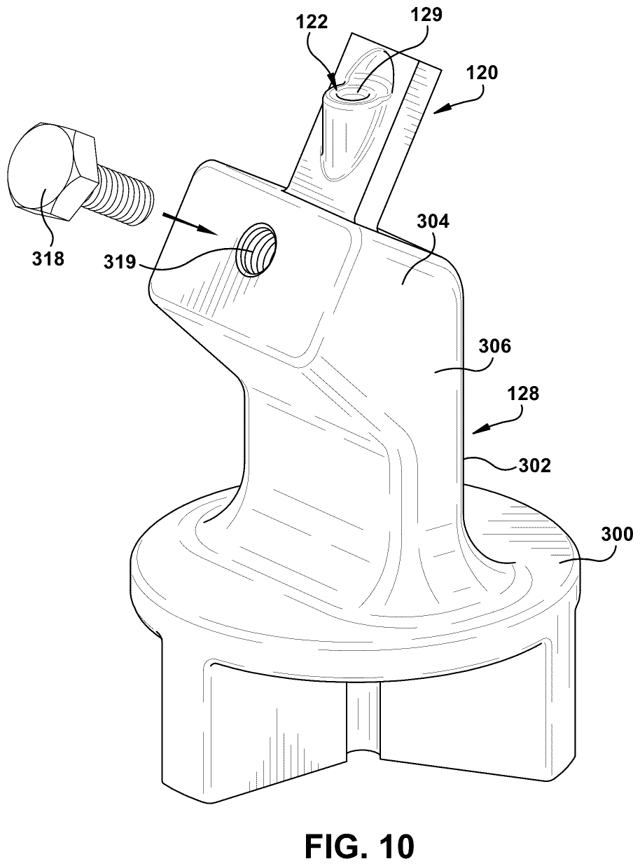

FIG. 8 shows a cross-sectional view and FIGS. 9-10 show perspective views of feature test coupon 120 in fixture 128 for inspection system 130 (FIG. 7), according to various embodiments of the disclosure. Referring to FIGS. 2, 3 and 8-10, fixture 128 may include a base 300, a stem 302 extending from base 300 and a mount 304 on an end 306 of stem 302 located distal from base 300. Base 300 is configured in any now known or later developed fashion to mount to 3D motion control system 310 of inspection system 130 (FIG. 7). Stem 306 extends from base 300 any distance to position mount 304 and, more particularly, feature test coupon 120 to maintain test feature 122 on within a focal field 312 (FIG. 7) of inspection system 130 (FIG. 7) as fixture 128 is moved through the predefined inspection path. Stem 306 may vary in length depending on, among other things, the size, shape and/or position of test feature 122, feature test coupon 120, 3D motion control system 310; and/or the size of inspection system 130. For example, stem 302 is shorter in FIG. 10 than in FIGS. 8-9. As will be described herein, the method may alternatively include configuring feature test coupon 120 including test feature 122 and fixture 128 for holding feature test coupon 120 relative to inspection system 130 (FIG. 7) to maintain test feature 122 within focal field 312 of inspection system 130 (FIG. 7) as fixture 128 is moved through a predefined inspection path. As will be further described herein, focal field 312 includes a three-dimensional window in which the inspection system (e.g., microscope, micro computed tomography (CT), very fine blue-light scan) can resolve into geometry as it passes along its focal Z-axis. Inspection system 130 (FIG. 7) gathers X and Y data points as the focus of the system observes the geometry of fixture 128, traveling down the Z axis allows for the third dimension as the focus contacts more X and Y data points in new depths.

As shown in FIGS. 3 and 8, fixture 128 also includes mating element 126 in mount 304 configured to receive in mating element 124 of feature test coupon 120. In the example shown, mating element 126 in mount 304 includes an opening configured to receive mating element 124 of feature test coupon 120. It will be recognized that the male-female mating relationship may be reversed. Fixture 128 may also include any now known or later developed manner of retainer member 318 configured of selectively hold feature test coupon 120 and mount 304 together. In the example shown, retainer member 318 includes a threaded fastener threadable into a mating hole 319 in mount 304 to engage/clamp feature test coupon 120 in mount 304. As will be recognized, retainer member 318 may take a variety of alternative forms, e.g., spring loaded member, a pinned member, etc.

Mount 304 of fixture 128 and feature test coupon 120 can be arranged to present test feature 122 in any desired manner for inspection. In the non-limiting example, test feature 122 on feature test coupon 120 and mating element 126 on mount 304 of fixture 128 are collectively angled to present first portion 129 of test feature 122, e.g., the opening in the nozzle, vertically when fixture 128 is mounted into 3D motion control system 310 of inspection system 130 in a neutral position, i.e., with base 300 of fixture 128 horizontally arranged. It will be recognized that a wide variety of different arrangements are possible, e.g., depending on the test feature 122 or portion thereof to be inspected.

Returning to the method and the flow diagram of FIG. 4, process P14 includes inspecting test feature 122 on feature test coupon 120 using inspection system 130 (FIG. 7). Process P14 also includes determining whether a defect exists in test feature 122 on feature test coupon 120. The presence of a defect may be determined using any now known or later developed technique using inspection system 130 (FIG. 7) and/or any other known analysis system for data obtained by inspection system 130 (FIG. 7). In some non-limiting examples, it may be determined that the opening in test feature 122 has an incorrect dimension such as an incorrect inner diameter; test feature 122 has an incorrect or poor surface roughness; portion of test feature 122 did not print in a required shape (e.g., it is out of round, has incorrect radius of curvature, has an incorrect transition between surfaces, etc.); test feature 122 is out of tolerance positioning; and/or test feature 122 has material abnormalities; etc.

In process P16, in response to a defect being detected in test feature 122 on feature test coupon 120, i.e., YES at process P14, a parameter is adjusted to remove the defect. The parameter(s) may be any now known or later developed operating parameter(s) of AM system 210 (FIG. 5) such as but not limited to: printing parameters in AM system 210 (FIG. 5) such as but not limited to print orientation, laser scanning speed, layer thickness, delay between layers, materials used. Alternatively, or in addition thereto, the parameter adjustment may be within feature 104 (and test feature 122 replica thereof) such as but not limited to: shape, size, location, orientation, etc.

Otherwise, in process P18, in response to a defect not being detected in test feature 122 on feature test coupon 120, i.e., NO at process P14, the method ends.

The method shown in FIG. 4 may also include repeating the steps of additive manufacturing on AM system 210 (FIG. 5) per process P10, and the mounting per process P12, and the inspecting per process P14 using an adjusted test feature 122. That is, per the arrows in FIG. 4, the method may be repeated after parameter(s) of AM system 210 (FIG. 5) and/or feature 104 is adjusted to remove the defect. Hence, the method shown in FIG. 4 also includes a (second) additively manufacturing step performed with parameter(s) of AM system 210 (FIG. 5) and/or feature 104 being different from the (first) additively manufacturing step. The second additively manufacturing step manufactures feature test coupon 120 including an adjusted test feature 122, i.e., based on the different parameter(s). In this case, the adjusted test feature on a new feature test coupon 120 is additively manufactured and analyzed to ensure the defect has been corrected.

The method of FIG. 4 may also be conducted for a plurality of the feature test coupons 120 (FIG. 2). In this case, process P10 includes (first) additively manufacturing the plurality of feature test coupons 120 (FIG. 2) as shown, for example, in FIG. 2. Test feature 122 on each of the plurality of feature test coupons 120 may have a different characteristic than test feature 122 on each other of plurality of feature test coupons 120. For example, a different surface roughness may be used on each different feature test coupon 120 so they can be evaluated. In this manner, different arrangements or iterations of test features 122 may be inspected and/or tested in bulk, without necessitating re-creation of the fixture, inspection system 130 (FIG. 7) program or automated inspection path. In process P14, the inspecting includes inspecting each test feature 122 on plurality of feature test coupons 120 using inspection system 130 (FIG. 7). Process P14 may be repeated for each feature test coupon 120. Process P16 includes adjusting parameter(s) of AM system 210 (FIG. 5) and/or feature 104 to remove a defect in response to the defect being detected in test feature 122 on at least one of the plurality of feature test coupons 120 (FIG. 3). The adjustments may include any described previously herein.

The method shown in FIG. 4 also includes a (second) additive manufacturing step performed with parameter(s) of AM system 210 (FIG. 5) and/or feature 104 being different from the (first) additively manufacturing step. The second additively manufacturing step manufactures feature test coupon 120 including an adjusted test feature, i.e., based on the different parameter(s).

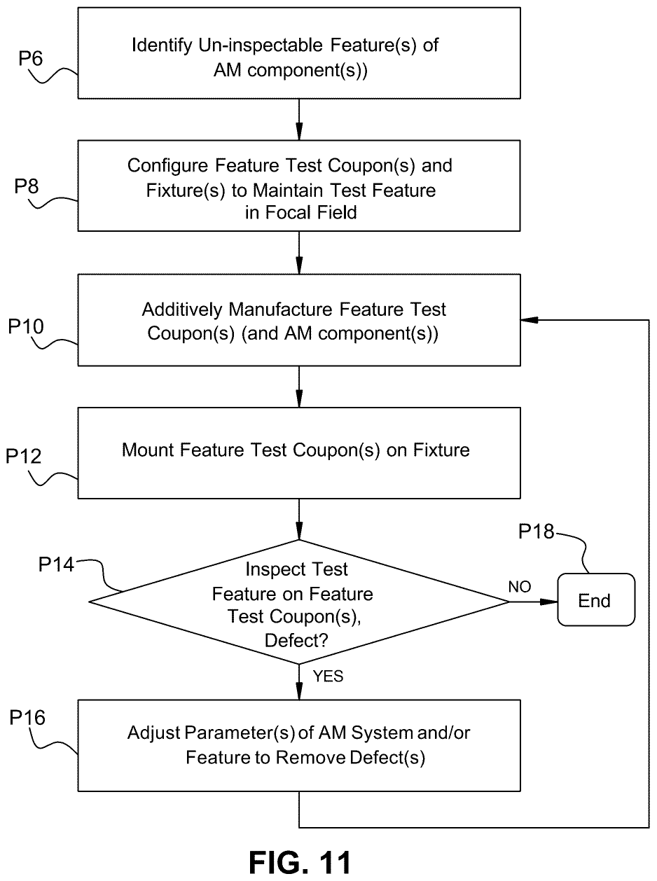

FIG. 11 shows a flow diagram of a method of inspecting feature 104 (FIG. 1) of AM component 100, according to other embodiments of the disclosure. The method of FIG. 11 includes two precursor processes, P6 and P8, to the method described herein relative to FIG. 4. Otherwise, the method of FIG. 11 is substantially similar to that of FIG. 4 relative to processes P10-P18 as described herein.

With reference to FIGS. 1, 3 and 7-11, in optional process P6, feature 104 of AM component 100 formed by AM system 210 (FIG. 5) that is un-inspectable is identified. In certain embodiments, features 104 may be located within AM component 100, i.e., within exterior surface 112 of AM component 100. In other embodiments, features 104 may be very small on AM component 100. However, the un-inspectable feature 104 can take any form described herein. Feature 104 so identified may include any feature described herein benefiting from the methods described herein. Feature 104 may be identified using any now known or later developed solution, e.g., user selection, modeling, empirical data, etc. The extent of feature 104 that forms test feature 122 can be user selected to ensure the desired data from the inspection can be obtained.

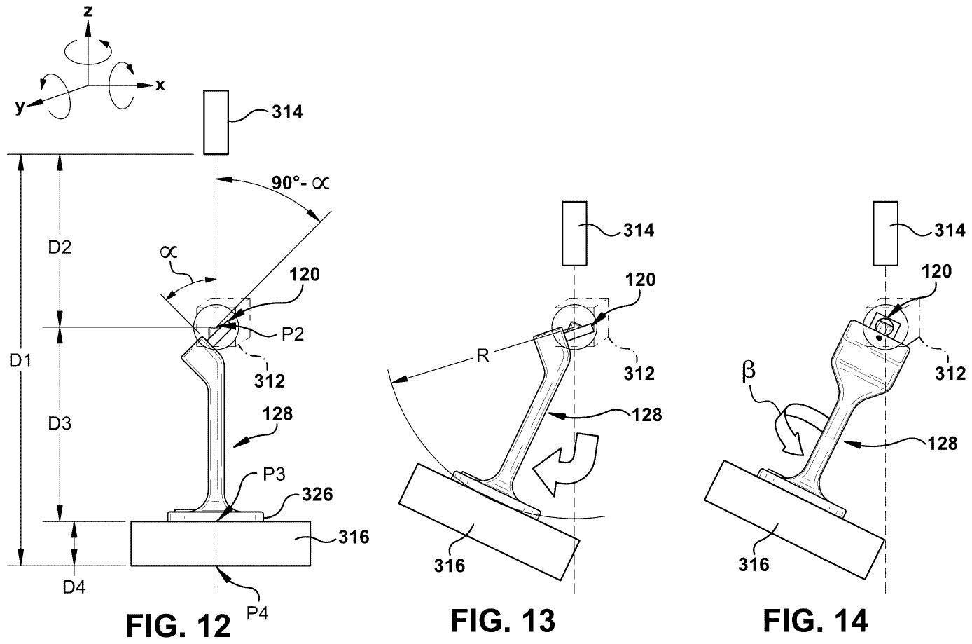

In process P8, feature test coupon 120 including test feature 122 that is substantially a replica of feature 104 of AM component 100 and fixture 128 for holding feature test coupon 120 relative to inspection system 130 are configured to maintain test feature 122 on feature test coupon 120 within focal field 312 of inspection system 130 as fixture 128 (and also, test feature 122) is moved through a predefined inspection path. As noted, focal field 312 includes a three-dimensional window in which focusing of any inspection element of inspection system 130 is not required. As noted, and as shown by the legend in FIG. 7, the predefined inspection path may include at least one linear movement in at least one of an X, Y and Z direction, tilt around at least one of an X and Y axis, and rotation around a Z axis. FIGS. 12-14 show side views of fixture 128 with feature test coupon 120 and inspection head 314 and platen 316 of inspection system 130 according to embodiments of the disclosure. The configuration can consider a wide range of attributes of size, shape, motion path(s), etc., of fixture 128, feature test coupon 120 and/or parts of inspection system 130. A non-comprehensive list of attributes to be considered in the configuration may include: a total height D1 (i.e., inspection head 314 to bottom of inspection system platen (fixturing jig) 316); focal length D2 (inspection head 314 to focal field 312); fixture 128 height D3 (bottom base 300/top of platen clamp 316 to assembled position of test feature 122); platen 316 thickness D4; rotational radius of platen R; feature test coupon angle α; rotational angle β; and/or feature test coupon 120 shape and/or dimensions. In most cases, fixture height D3 will equal rotational radius R to maintain feature test coupon 120 aligned on an optic's axis of inspection head 314. Most inspection systems 130 will raise and/or lower inspection head 314 along the Z-axis to make distance to inspection head 314 variable, adjusting focus through various Z-heights of the inspected object to measure three-dimensionally. Focal length D2 is consistent.

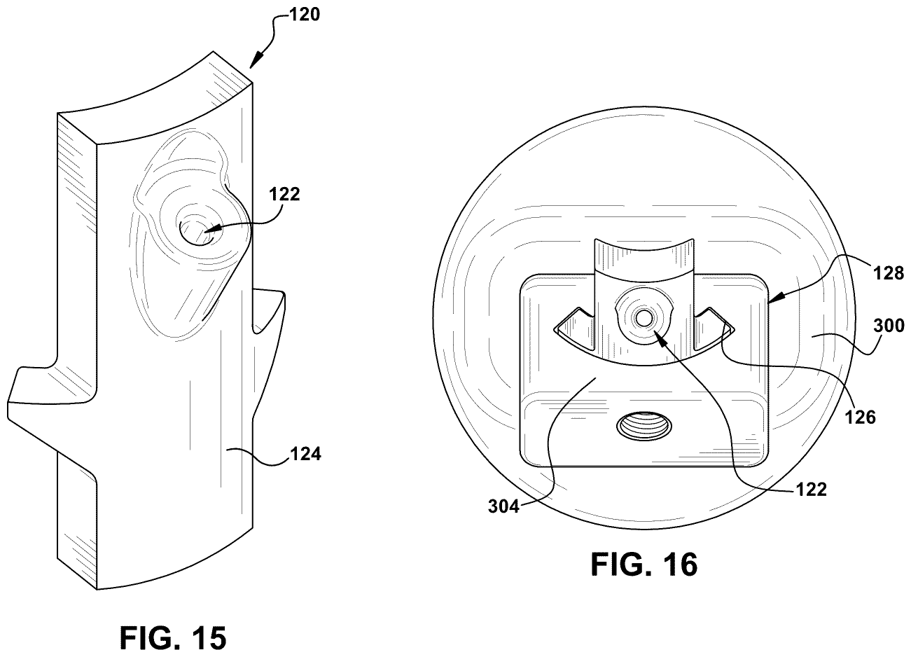

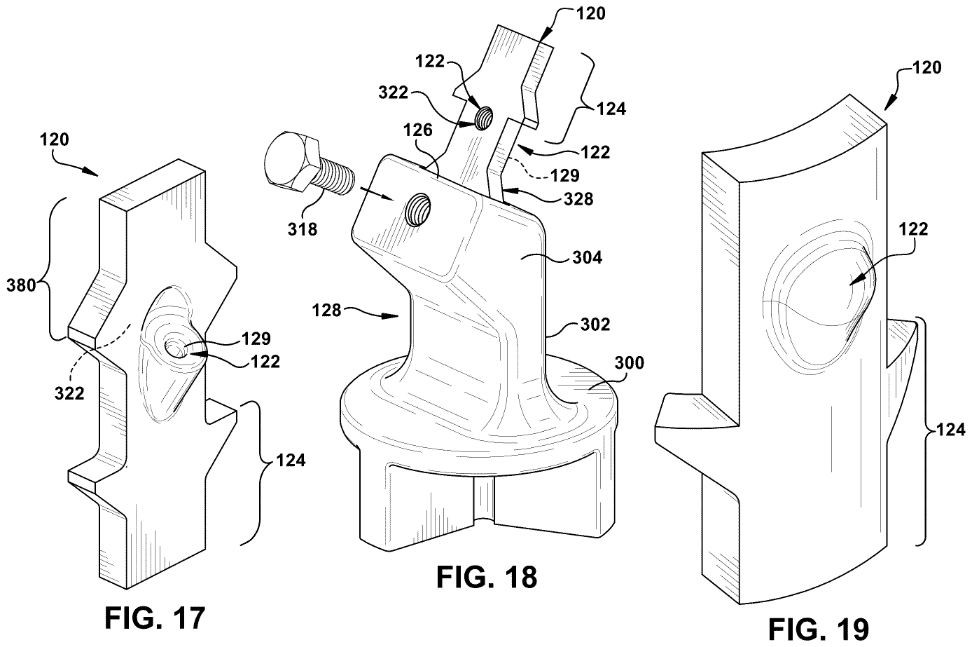

With further regard to feature test coupon 120 shape and/or dimensions, FIGS. 15 and 16 shows perspective views of feature test coupon 120 including a replica of feature 104 to be inspected, according to other embodiments of the disclosure. FIGS. 15 and 16 show other shapes and/or configurations that may aid in configuring feature test coupon 120 to maintain test feature 122 on feature test coupon 120 within focal field 312 (FIGS. 12-14) of inspection system 130 as fixture 128 (and also, test feature 122) is moved through a predefined inspection path. FIGS. 15 and 16 show a feature test coupon 120 with mating element 124 (or a body thereof) having a curved cross-section and with mating element 126 in mount 304 of fixture 128 having an identical curvature. FIGS. 17 and 18 show a feature test coupon 120 that includes another mating element 320 configured to removably mate with mating element 126 of mount 304 of fixture 128 in a manner to present a second, different portion 322 of test feature 122 for the inspecting. In the example shown, second different portion 322 includes a back side of the fuel nozzle and, in particular, the opening thereof. In this manner, feature test coupon 120 is capable of providing inspection of more than one portion of test feature 122. In other words, mating elements 126, 324 of feature test coupon 120 make it reversible. Mating elements 124, 126, and/or 324 are printable with feature test coupon 120 and allow for repeatable z-height control respective to focus field 312 (FIGS. 12-14) when placed on fixture 128. The reversible nature of feature test coupon 120, i.e., flipping in fixture 128, also afford compound analysis on inspection systems 130 that may that only permit 90° tilt. FIG. 19 shows a feature test coupon 120 that illustrates another type of test feature 122 than a hole as in FIG. 17.

Embodiments of the disclosure provide various technical and commercial advantages, examples of which are discussed herein. The methods provide for inspecting otherwise un-inspectable, additively manufactured features. The methods are also advantageous for small or fine features that are not easily inspectable on a large AM component. The feature test coupon may be standardized and fits into a specifically designed fixture that places the feature within the focal field of the inspection system. The inspection may include, for example, optical surface roughness and shape measuring, with an automated multi-scan inspection system that combines multiple fine surface roughness scans into a high resolution, single geometry output for development and ongoing quality checks. Isolating the small features that are not otherwise inspectable onto a universal feature test coupon also increases the abilities of the non-destructive inspection over similar blue-light technology. More particularly, the fidelity of the resolution is an order of magnitude more precise than blue-light technology. An automated multi-scan inspection system allows for consistent, reliable results at the resolution of a surface roughness measurement. The methods also allow for rapid feedback thus allowing for both operational parameter and geometric parameter improvements. The methods can be used regardless of component or feature geometry, materials, or AM system used. The methods also allow rapid design iterations without needing to create, fixture and inspect an entire AM component every time. The small size and ease of additive manufacture of the feature test coupons remove practically all limitations to the number of design iterations that can be printed on the same AM system configuration. The efficient, automated and consistent inspections provide quantitative results for both the component (short-term) and the machine (long-term) in which used, e.g., a turbomachine. The universal test feature also increases ease in manufacturability, ease in creating multiple variations for testing, and the efficiency of trialing multiple variations in one-to-one comparisons.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise. “Approximately” or “about,” as applied to a particular value of a range, applies to both end values and, unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiments were chosen and described in order to best explain the principles of the disclosure and the practical application of the technology and to enable others of ordinary skill in the art to understand the disclosure for contemplating various modifications to the present embodiments, which may be suited to the particular use contemplated.

What is claimed is:

1. A method, comprising:
first additively manufacturing a feature test coupon including a test feature, the test feature being a replica of a feature of an additively manufactured (AM) component formed by an additive manufacturing system that is un-inspectable;
mounting the feature test coupon on a fixture for holding the feature test coupon relative to an inspection system;
inspecting the test feature on the feature test coupon using the inspection system; and
in response to a defect being detected in the test feature on the feature test coupon, adjusting a parameter of at least one of the additive manufacturing system or the feature to remove the defect.

2. The method of claim 1, wherein the feature is un-inspectable from an exterior of an additively manufactured (AM) component formed by the additive manufacturing system.

3. The method of claim 1, further comprising:
identifying the feature of the AM component that is un-inspectable from an exterior of the AM component to be inspected; and
configuring the feature test coupon including the test feature and the fixture for holding the feature test coupon relative to the inspection system to maintain the test feature on the feature test coupon within a focal field of the inspection system as the fixture is moved through a predefined inspection path.

4. The method of claim 3, wherein the focal field includes a three-dimensional window in which focusing of any inspection element of the inspection system is not required.

5. The method of claim 3, wherein the feature test coupon is configured to orient the test feature for inspection of a set of predefined critical-to-quality (CTQ) parameters.

6. The method of claim 3, wherein the feature test coupon includes a first mating element configured to removably mate with a second mating element of the fixture in a manner to present a first portion of the test feature for the inspecting.

7. The method of claim 5, wherein the feature test coupon includes a third mating element configured to removably mate with the second mating element of the fixture in a manner to present a second, different portion of the test feature for the inspecting.

8. The method of claim 3, wherein the predefined inspection path includes at least one linear movement in at least one of an X, Y and Z direction, tilt around at least one of an X and Y axis, and rotation around a Z axis.

9. The method of claim 1, wherein first additively manufacturing the feature test coupon includes additively manufacturing a plurality of the feature test coupons, wherein the test feature on each of the plurality of feature test coupons has a different characteristic than the test feature on each other of the plurality of feature test coupons, and
wherein the inspecting includes inspecting each test feature on the plurality of feature test coupons using the inspection system, and
wherein the adjusting includes, in response to the defect being detected in the test feature on at least one of the plurality of feature test coupons, adjusting the parameter to remove the defect.

10. The method of claim 1, further comprising a second additive manufacturing step performed with the parameter of the at least one of the additive manufacturing system or the feature being different from the first additively manufacturing step, the second additively manufacturing step manufacturing the feature test coupon including an adjusted test feature.

11. The method of claim 1, further comprising repeating the additive manufacturing on the additive manufacturing system, mounting and inspecting steps using an adjusted test feature.

12. The method of claim 1, wherein the test feature that is un-inspectable from the exterior of the AM component is located within the AM component.

13. A method, comprising:
identifying a feature of an additively manufactured (AM) component formed by an additive manufacturing system that is un-inspectable;
configuring a feature test coupon including a test feature that is substantially a replica of the feature of the AM component and a fixture for holding the feature test coupon relative to an inspection system to maintain the test feature on the feature test coupon within a focal field of the inspection system as the fixture is moved through a predefined inspection path;
first additively manufacturing the feature test coupon on the additive manufacturing system;
mounting the fixture relative to the inspection system and the feature test coupon on the fixture;
inspecting the test feature on the feature test coupon using the inspection system and moving the fixture along the predefined inspection path to identify a defect in the test feature on the feature test coupon, wherein the test feature on the feature test coupon remains within the focal field of the inspection system as the fixture is moved through the predefined inspection path; and
in response to a defect being detected in the test feature on the feature test coupon, adjusting a parameter of at least one of the additive manufacturing system or the feature to remove the defect.

14. The method of claim 13, wherein the feature is located within the AM component.

15. The method of claim 13, wherein the focal field includes a three-dimensional window in which focusing of any inspection element of the inspection system is not required.

16. The method of claim 13, wherein the additive manufacturing of the feature test coupon is configured to orient the test feature for inspection of a set of predefined critical-to-quality (CTQ) parameters.

17. The method of claim 13, wherein the feature test coupon includes a first mating element configured to removably mate with a second mating element of the fixture in a manner to present a first portion of the test feature for the inspecting.

18. The method of claim 17, wherein the feature test coupon includes a third mating element configured to removably mate with the second mating element of the fixture in a manner to present a second, different portion of the test feature for the inspecting.

19. The method of claim 13, wherein the predefined inspection path includes at least one linear movement in at least one of an X, Y and Z direction, tilt around at least one of an X and Y axis, and rotation around a Z axis.

20. The method of claim 13, further comprising a second additively manufacturing step performed with the parameter of the at least one of the additive manufacturing system or the feature being different from the first additively manufacturing step, the second additively manufacturing step manufacturing the feature test coupon including an adjusted test feature.

21. The method of claim 13, further comprising repeating the first additively manufacturing step, mounting and inspecting using an adjusted test feature.
